(19) United States
(12) Patent Application Publication
(10) Pub. No.: US 2026/0072057 A1
(43) Pub. Date: Mar. 12, 2026

(54) METHOD TO DETECT FAILED PIEZOELECTRIC TRANSDUCER IN ULTRASOUND APPLICATIONS

(71) Applicant: STMicroelectronics International N.V., Geneva (CH)

(72) Inventor: John WHALEN, Manchester, NH (US)

(21) Appl. No.: 18/828,145

(22) Filed: Sep. 9, 2024

(51) Int. Cl. G01R 15/20; A61B 8/00; G01N 29/02; G01N 29/24; G01R 19/00

(52) U.S. Cl. CPC G01R 15/202; G01N 29/02; G01N 29/2437; G01R 19/0092; A61B 8/4427; A61B 8/58

(57) ABSTRACT

Apparatuses, systems, and methods for detecting failed piezoelectric transducers in ultrasound applications. An exemplary method includes determining a current consumed by at least a piezoelectric transducer element in a system via a sensor positioned to sense current in a return path from the piezoelectric transducer element; and determining an operational state of the piezoelectric transducer element based at least in part on the consumed current.

### TECHNOLOGICAL FIELD

Example embodiments of the present disclosure relate generally to systems and methods for detecting failed piezoelectric transducers in ultrasound applications.

### BACKGROUND

Ultrasound tools are used to assess, in varying degrees, the composition of both organic and inorganic materials. Ultrasound tools are prolific diagnostic tools, which are used for both industrial and clinical applications. For instance, ultrasound tools are used in various applications to assess the composite structural integrity of machine components, such as a jet airliner outer skin. Additionally, ultrasound tools are utilized as part of quality control systems, such as for plastics and joints. Moreover, ultrasound tools are used in animal diagnostic clinics, as well as for both diagnostic and therapeutic human applications.

To improve user experience, ultrasound diagnostic tools have evolved from being relatively large stationary devices to relatively small mobile devices. For instance, mobile ultrasound devices may be moved to a patient rather than the patient being moving to a stationary ultrasound device. While mobile ultrasound devices may provide for an improved user experience, such devices may be more susceptible to impacts, such as from being dropped. Some ultrasound diagnostic tools include piezoelectric elements constructed from relatively fragile materials. Consequently, a piezoelectric element within an ultrasound device may sustain damage from an impact. Such damage may cause the piezoelectric element to fail.

New systems and methods for detecting failed piezoelectric elements are needed. The inventors have identified numerous areas of improvement in the existing technologies and processes, which are the subjects of embodiments described herein. Through applied effort, ingenuity, and innovation, many of these deficiencies, challenges, and problems have been solved by developing solutions that are included in embodiments of the present disclosure, some examples of which are described in detail herein.

### BRIEF SUMMARY

Various embodiments described herein relate to systems and methods for detecting failed piezoelectric transducers in ultrasound applications.

In accordance with some embodiments of the present disclosure, an example method is provided. In some embodiments, the method comprises: determining a current consumed by at least a piezoelectric transducer element in a system via a sensor positioned to sense current in a return path from the piezoelectric transducer element; and determining an operational state of the piezoelectric transducer element based at least in part on the current consumed by the piezoelectric transducer element.

In some embodiments, determining the operational state of the piezoelectric transducer element comprises determining that the operational state of the piezoelectric transducer element is a sub-performance state based at least in part on the current failing to satisfy one or more criteria associated with a baseline performance of the piezoelectric transducer element.

In some embodiments, the method comprises obtaining one or more baseline current measurements associated with the piezoelectric transducer element, wherein the one or more criteria are based at least in part on the one or more baseline current measurements.

In some embodiments, a baseline current measurement of the one or more baseline current measurements comprise a current measurement obtained at a fixed voltage, a fixed operating frequency, and a fixed temperature.

In some embodiments, the method comprises determining, based at least in part on the one or more baseline current measurements, a lifetime drift and an expected temperature variation associated with current consumption by the piezoelectric transducer element, wherein the one or more criteria are based at least in part on the lifetime drift and the expected temperature variation.

In some embodiments, the one or more criteria are further based at least in part on at least one of: an interface associated with the piezoelectric transducer element or a position of the piezoelectric transducer element within an array.

In some embodiments, the return path comprises an electrical path between the piezoelectric transducer element and a ground terminal.

In some embodiments, the sensor comprises a shunt resistor, a Hall effect sensing device, a giant magneto resistance (GMR) sensing device, or a tunnel magneto resistance (TMR) sensing device.

In some embodiments, the sensor comprises the Hall effect sensing device.

In some embodiments, the piezoelectric transducer element is one of a plurality of piezoelectric transducer elements within the system, and the method further comprises: determining a respective current consumed by each piezoelectric transducer element of the plurality of piezoelectric transducer elements via one or more sensors positioned to sense current in one or more return paths from the plurality of piezoelectric transducer elements, the one or more sensors comprising at least the sensor and the one or more return paths comprising at least the return path.

In some embodiments, the system comprises an ultrasound system.

In accordance with some embodiments of the present disclosure, an example system is provided. In some embodiments, the example system comprises a piezoelectric transducer element; a sensor positioned to sense current in a return path from the piezoelectric transducer element; and a processor configured to determine an operational state of the piezoelectric transducer element based at least in part on the current sensed in the return path.

In some embodiments, the processor is further configured to determining that the operational state of the piezoelectric transducer element is a sub-performance state based at least in part on the current failing to satisfy one or more criteria associated with a baseline performance of the piezoelectric transducer element.

In some embodiments, the processor is further configured to obtain one or more baseline current measurements associated with the piezoelectric transducer element, wherein the one or more criteria are based at least in part on the one or more baseline current measurements.

In some embodiments, the return path comprises an electrical path between the piezoelectric transducer element and a ground terminal.

In some embodiments, the sensor comprises a shunt resistor, a Hall effect sensing device, a giant magneto resistance (GMR) sensing device, or a tunnel magneto resistance (TMR) sensing device.

In accordance with some embodiments of the present disclosure, an example apparatus is provided. In some embodiments, the example apparatus comprises at least one processor; and at least one memory having computer program code stored thereon that, in execution with the at least one processor, causes the apparatus at least to: determine a current consumed by at least a piezoelectric transducer element in a system via a sensor positioned to sense current in a return path from the piezoelectric transducer element; and determine an operational state of the piezoelectric transducer element based at least in part on the current consumed by the piezoelectric transducer element.

In some embodiments, the computer program code, in execution with the at least one processor, causes the apparatus to determine that the operational state of the piezoelectric transducer element is a sub-performance state based at least in part on the current failing to satisfy one or more criteria associated with a baseline performance of the piezoelectric transducer element.

In some embodiments, the computer program code, in execution with the at least one processor, causes the apparatus to obtain one or more baseline current measurements associated with the piezoelectric transducer element, wherein the one or more criteria are based at least in part on the one or more baseline current measurements.

In some embodiments, the return path comprises an electrical path between the piezoelectric transducer element and a ground terminal.

The above summary is provided merely for purposes of summarizing some example embodiments to provide a basic understanding of some aspects of the disclosure. Accordingly, it will be appreciated that the above-described embodiments are merely examples and should not be construed to narrow the scope or spirit of the disclosure in any way. It will also be appreciated that the scope of the disclosure encompasses many potential embodiments in addition to those here summarized, some of which will be further described below.

### BRIEF SUMMARY OF THE DRAWINGS

Having thus described some example embodiments of the present disclosure in general terms, reference will now be made to the accompanying drawings, which are not necessarily drawn to scale, and wherein:

FIG. 1 illustrates an exemplary block diagram of a system for detecting failed piezoelectric transducers in ultrasound applications in accordance with at least one embodiment of the present disclosure;

FIG. 2 illustrates an exemplary block diagram of an ultrasound system configured for detecting failed piezoelectric transducers in ultrasound applications in accordance with at least one embodiment of the present disclosure;

FIG. 3 illustrates an exemplary flowchart of operations that support systems and methods detecting failed piezoelectric transducers in ultrasound applications in accordance with at least one embodiment of the present disclosure; and

FIG. 4 illustrates an exemplary block diagram of a device that support systems and methods for detecting failed piezoelectric transducers in ultrasound applications in accordance with at least one embodiment of the present disclosure.

### DETAILED DESCRIPTION

Some embodiments of the present disclosure will now be described more fully herein with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout.

As used herein, the term “comprising” means including but not limited to. The term comprising should be interpreted in the manner it is typically used in the patent context. Use of broader terms such as comprises, includes, and having should be understood to provide support for narrower terms such as consisting of, consisting essentially of, and comprised substantially of.

The phrases “in various embodiments,” “in one embodiment,” “according to one embodiment,” “in some embodiments,” and the like generally mean that the particular feature, structure, or characteristic following the phrase may be included in at least one embodiment of the present disclosure and may be included in more than one embodiment of the present disclosure. Such phrases do not necessarily refer to the same embodiment.

The word “example” or “exemplary” is used herein to mean “serving as an example, instance, or illustration.” Any implementation described herein as “exemplary” is not necessarily to be construed as preferred or advantageous over other implementations.

If the specification states a component or feature “may,” “can,” “could,” “should,” “would,” “preferably,” “possibly,” “typically,” “optionally,” “for example,” “often,” or “might” (or other such language) be included or have a characteristic, that a specific component or feature is not required to be included or to have the characteristic. Such a component or feature may be optionally included in some embodiments or it may be excluded.

The use of the term “circuitry” as used herein with respect to components of a system or an apparatus should be understood to include particular hardware configured to perform the functions associated with the particular circuitry as described herein. The term “circuitry” should be understood broadly to include hardware and, in some embodiments, software for configuring the hardware. For example, in some embodiments, “circuitry” may include processing circuitry, communications circuitry, input/output circuitry, and the like. In some embodiments, other elements may provide or supplement the functionality of particular circuitry.

The term “sense current” is used herein to mean measure a current or otherwise determine a current based on measurements performed using a device. Such a device is referred to herein as a sensor. The current may be sensed using a device configured to measure current flow. Additionally, or alternatively, the current may be sensed using a device configured to measure one or more other properties that are indicative of current flow. For example, the current may be sensed using a device configured to measure the magnetic field produced by flowing current. Non-limiting examples of devices configured to sense current based on measuring the magnetic field (or properties thereof) include a Hall-effect sensor, a giant magneto resistance (GMR) sensor, and a tunnel magneto resistance (TMR) sensor. In some other examples, the current may be sensed using a device configured to measure the voltage drop across a resistor. A non-limiting example of such a device includes a shunt resistor.

The term “current consumed by a piezoelectric transducer element,” and the like, is used herein to mean the current drawn by a piezoelectric transducer element. In some instances, the current drawn by a piezoelectric transducer element (or a portion thereof) is converted into one or more signals. In other words, a piezoelectric transducer element may consume current to generate one or more signals, such as ultrasonic signals for ultrasound applications.

The term “return path” is used herein to mean an electrical path that current takes after exiting a node. In a non-limiting example, the return path includes an electrical path that current takes back to its source after exiting a node. In another non-limiting example, the return path includes an electrical path that current takes to a ground terminal after exiting a node. The term “node” is used herein to mean a point of connection (i.e. junction) between two or more circuit elements.

The term “operational state of a piezoelectric transducer element,” and the like, is used herein to mean a level of performance associated with the operation of a piezoelectric transducer element. In one non-limiting example, the operational state includes a baseline performance state in which the level of performance associated with the operation of a piezoelectric transducer element is consistent with a baseline performance (i.e., normal performance) of the piezoelectric transducer element. In a non-limiting example, the level of performance associated with the operation of the piezoelectric transducer element is consistent with the baseline performance of the piezoelectric transducer element if a current determined for the piezoelectric transducer element satisfies one or more criteria associated with the baseline performance of the piezoelectric transducer element. In some embodiments, the current may satisfy the one or more criteria if a difference between the value of the current and the value of a predicted current is less than a threshold. In some such embodiments, the predicted current corresponds to a current that the piezoelectric transducer element is expected to draw when operating in accordance with the baseline performance. In some non-limiting examples, the predicted current and/or the threshold may be based on one or more baseline current measurements obtained for the piezoelectric element, in which each baseline current measurement is obtained at a fixed voltage (e.g., a fixed high voltage), a fixed operating frequency, a fixed temperature, and with one or more known piezoelectric transducer element characteristics. Non-limiting examples of piezoelectric transducer characteristics include a type of material included in the piezoelectric transducer element, the piezoelectric driver circuitry configured to output current to the piezoelectric transducer element, the circuitry associated with the sensor, an interface medium associated with the piezoelectric transducer element (e.g., a printed circuit board (PCB), a cable, a flex circuit), and/or a position of the piezoelectric transducer element within an array. In some embodiments, the predicted current and/or threshold is based on multiple baseline current measurements obtained across a range of ambient temperatures. In some embodiments, a lifetime drift and/or an expected temperature variation may be determined for the piezoelectric transducer element based on one or more baseline current measurements. In some such embodiments, the predicted current and/or threshold may be based on the lifetime drift and/or the expected temperature variation.

In some other non-limiting examples, the operational state includes a sub-performance state in which the level of performance associated with the operation of a piezoelectric transducer element is inconsistent with a baseline performance (i.e., normal performance) of the piezoelectric transducer element. In a non-limiting example, the level of performance associated with the operation of the piezoelectric transducer element is inconsistent with the baseline performance of the piezoelectric transducer element if a current determined for the piezoelectric transducer element fails to satisfy one or more criteria associated with a baseline performance of the piezoelectric transducer element. In some embodiments, the current may fail to satisfy the one or more criteria if a difference between the value of the current and the value of the predicted current is greater than a threshold.

The term “baseline performance of a piezoelectric transducer element,” and the like, is used herein to mean a performance of the piezoelectric transducer element when the piezoelectric transducer element is functioning within a tolerance of a system, such that data collected by the system from the piezoelectric transducer element is sufficiently accurate. In some non-limiting examples, a piezoelectric transducer element may exhibit baseline performance when the piezoelectric transducer element is undamaged or otherwise intact.

The term “sub-performance of a piezoelectric transducer element,” and the like, is used herein to mean a performance of the piezoelectric transducer element when the piezoelectric transducer element is functioning outside of a tolerance of a system, such that data collected by the system from the piezoelectric transducer element is insufficiently accurate. In some non-limiting examples, a piezoelectric transducer element may exhibit sub-performance when the piezoelectric transducer element is damaged or otherwise impaired.

The term “lifetime drift associated with current consumption by a piezoelectric transducer element,” and the like, is used herein to mean a change in the value of the current drawn by the piezoelectric element over a particular operating time of the piezoelectric element.

The term “expected temperature variation associated with current consumption by a piezoelectric transducer element,” and the like, is used herein to mean a predicted change in the value of the current drawn by the piezoelectric element as a function of the ambient temperature.

### Overview

Ultrasound tools are used to assess, in varying degrees, the composition of both organic and inorganic materials. Ultrasound tools are prolific diagnostic tools, which are used for both industrial and clinical applications. For instance, ultrasound tools are used in various applications to assess the composite structural integrity of machine components, such as a jet airliner outer skin. Additionally, ultrasound tools are utilized as part of quality control systems, such as for plastics and joints. Moreover, ultrasound tools are used in animal diagnostic clinics, as well as for both diagnostic and therapeutic human applications.

Ultrasound diagnostic tools have evolved from being relatively large stationary devices to relatively small handheld devices that are portable. For example, some handheld ultrasound devices may connect to a mobile device (e.g., a tablet or cell phone), which may be moved to a patient, rather than the patient being moving to a stationary device. As such, it is relatively commonplace for handheld ultrasound devices to be found in outpatient clinics, doctors'offices, and in ambulatory conditions. For example, smaller (handheld) ultrasound machines have been repeatedly refined for specific applications (e.g., medical indications) and have enabled clinicians to see what a subcutaneous tissue or bone construct is. Medical ultrasound tools are utilized to assess cardiac blood flow, tumor construct, guidance for catheter placement, human development during pregnancy and beyond. Ultrasound tools are also commonly used in industrial applications, such as for non-destructive testing to assess material integrity. A non-limiting example of such use includes aircraft fuselage testing utilizing composite materials, where traditional Eddy current tests cannot be utilized. While handheld ultrasound devices may provide for an improved user experience, such devices may be more susceptible to impacts, such as from being dropped. That is, handheld ultrasound devices may have an increased likelihood of being dropped, such as to the floor or onto another rigid surface. While some elements within an ultrasound tool may withstand the shock associated with a drop to the floor, ultrasound devices include piezoelectric elements that are relatively fragile and may be unable to withstand the shock. For example, some ultrasound tools include piezoelectric elements constructed from quartz crystals, ceramics, and ferroelectric materials. Such piezoelectric elements may sustain damage (e.g., may can crack or break) from impacts, such as those associated with a drop to the floor (or another rigid surface).

The behavior of an intact (e.g., normal) piezoelectric element is relatively predictable. For instance, piezoelectric material (e.g., crystal) of the piezoelectric element represents a fixed complex impedance. As such, a current consumed by the piezoelectric material at a fixed voltage, frequency, and temperature is predictable over the lifetime of the piezoelectric material. The complex impedance may be characterized as a fixed capacitance and resistance. As an illustrative example, the component values of the impedance may be found as 1 micro Farad (uF) for the capacitance, and 100 ohms for the resistance. In such an example, the consequent current may be 1 ampere (A) at a given frequency (e.g., 10 mega Hertz (MHz) and a given voltage (e.g., 60 volts (V) peak to peak).

In some instances, however, damage to a piezoelectric element may disable the piezoelectric effect on the piezoelectric element, which may cause the current consumed by the piezoelectric element to become unpredictable. For instance, if an ultrasound tool with a piezoelectric element (also referred to as a piezoelectric transducer element or, more simply, a crystal transducer) is dropped to the floor, and the piezoelectric element is damaged, the electrical characteristics of the piezoelectric element may change. For instance, the capacitance of the piezoelectric element may change with an impact (e.g., a sudden deceleration) from a drop to the floor.

In some instances, it may be relatively difficult to determine whether a piezoelectric element in a device, such as an ultrasound tool, is damaged. Some methods for detecting a damaged piezoelectric element include a user viewing an ultrasound image obtained using the ultrasound tool, identifying anomalies in the image, and reporting the anomalies to the equipment manufacturer (e.g., reporting to the equipment manufacturer that the ultrasound tool has failed in some manner). In other words, some methods for detecting damage to a piezoelectric element in an ultrasound tool may include a user identifying a failure of the ultrasound tool through use of the tool, such as during a patient procedure or for a material analysis. Consequently, the user (e.g., a clinician) may not know that a piezoelectric element in an ultrasound tool has been damaged (e.g., from a dropping incident) prior to using the ultrasound tool and/or until an image is displayed and an anomaly is observed within the image. In some cases, observed image anomalies that are due to a damaged piezoelectric crystal may be mistakenly attributed to anomalies associated with a patient (rather than the ultrasound tool itself), which may lead to delays for the patient and/or a misdiagnosis.

Various aspects of the present disclosure are directed to improved systems and methods for detecting failed piezoelectric transducers in ultrasound applications. In some embodiments, the present disclosure provides for systems and methods for detecting a failed piezoelectric transducer based sensing a current consumed by the piezoelectric transducer. For example, in accordance with one or more systems and/or methods of the present disclosure, the current consumed by a piezoelectric element may be measured multiple times (e.g., repeatedly). Because the impedance of an intact piezoelectric element is constant, the current consumed by the intact piezoelectric element at a fixed voltage, frequency, and temperature is also constant. Accordingly, if the multiple current measurements are performed while the piezoelectric element is intact, and consuming a predictable amount of current, the current measurements will be the same within a tolerance of the system. However, if a current measurement is performed while the piezoelectric element is damaged or otherwise impaired, the current measurement will change due to the piezoelectric element consuming more or less current than is predicted. In accordance with one or more systems and/or methods of the present disclosure, a failed piezoelectric transducer may be detected based sensing a current consumed by the piezoelectric transducer.

In accordance with one or more embodiments of the present disclosure, a piezoelectric driver circuit of a system may be modified to add one or more current sensing elements, which may determine the current consumed by one or more piezoelectric elements of the system. For example, the system may be, or may be included in, an ultrasound tool, such as may be used for diagnostic tests in clinical or industrial applications. The ultrasound tool may include one or more arrays of piezoelectric elements. That is, the ultrasound tool may include one or more piezoelectric transducer bulk arrays, such as one or more zirconate titanate (PZT) bulk array. The piezoelectric elements may be coupled to the piezoelectric driver circuit, such that the piezoelectric driver circuit may serve as a current source for the piezoelectric elements. For example, the piezoelectric elements may draw current from the piezoelectric driver circuit to generate one or more ultrasonic signals.

The current consumed by one or more of the piezoelectric elements may be measured at one or more locations within the system. For example, the current may be measured at the piezo driver output stage (e.g., in a forward path between the piezoelectric driver circuit and the one or more piezoelectric elements) and/or at one or more high voltage power supplies for the piezoelectric driver circuit. In some instances, however, the voltage going into the piezoelectric driver and/or the one or more piezoelectric elements is substantially higher than a common mode voltage of elements used for measuring the current, which may impact an accuracy of the current measurements. For example, if a relatively high voltage is divided down (e.g., to the common mode voltage), the accuracy and resolution of the current measurements may be reduced. The voltage in a return path from a piezoelectric element may be relatively close to the common mode voltage of the current measurement elements. For example, the voltage may be within a suitable range of the common mode voltage of the current measurement elements. In accordance with one or more embodiments of the present disclosure, to improve the resolution and accuracy of the current measurements, the current consumed by one or more of the piezoelectric elements may be measured in the return path from the one or more piezoelectric elements.

In some embodiments, the current sensing element may be included in series with one or more nodes (e.g., one or more nodes in the return path from one or more piezoelectric elements). In some embodiments, the current sensing element may be integrated onto an integrated circuit (IC). In some such embodiments, the current sensing element (e.g., a sensor) may be integrated into an IC with one or more operational amplifiers. In some embodiments, the current sensing element includes a shunt resistor, a Hall effect sensing device, a GMR sensing device, or TMR sensing device.

In some embodiments, a baseline (e.g., normal) current consumption may be determined (and recorded) for the one or more piezoelectric elements. The baseline current may correspond to the current determined in accordance with one or more parameters, such as a fixed high voltage, a fixed frequency, one or more known characteristics of the piezoelectric elements, a completed transmission medium to the piezoelectric elements (e.g., PBC, flex circuit, or cable), and/or a fixed ambient temperatures. In some embodiments, the baseline current may be determined across a range of ambient temperatures. In some embodiments, a respective baseline current may be determined for each piezoelectric element in the system (e.g., as there may be subtle differences associated with piezo placement within the array). In some embodiments, a suitable system tolerance level may be determined based on one or more baseline current measurements. The system tolerance level may be stored within the system. In some embodiments, the current consumed by one or more piezoelectric elements within the system may be measured and compared to a current associated with a control element (e.g., a piezoelectric element that is operating normally). In some embodiments, images collected via the ultrasound tool may be verified against control images (e.g., images associated with a baseline current consumption). That is, in some embodiments, the present disclosure provides for measuring and comparing currents between control elements, as well as verifying against respective images.

In some examples, the systems and methods for detecting failed piezoelectric elements in ultrasound applications, as described herein, may provide for improved self-diagnostics, improved imaging quality and assessment, reduced time per procedure, proactive replacement rather than reactive ultrasound device replacement, and improved functioning of a clinical or industrial environment due to less interruptions. For example, the present disclosure may improve clinical experiences for patients and reduce time associated with a technician assessing the integrity of a material, such as an aircraft fuselage during periodic integrity inspections.

### Exemplary Systems, Apparatuses, and Methods

The present disclosure provides for systems, apparatuses, and methods for detecting failed piezoelectric transducers in ultrasound applications, which may be implemented in some embodiments.

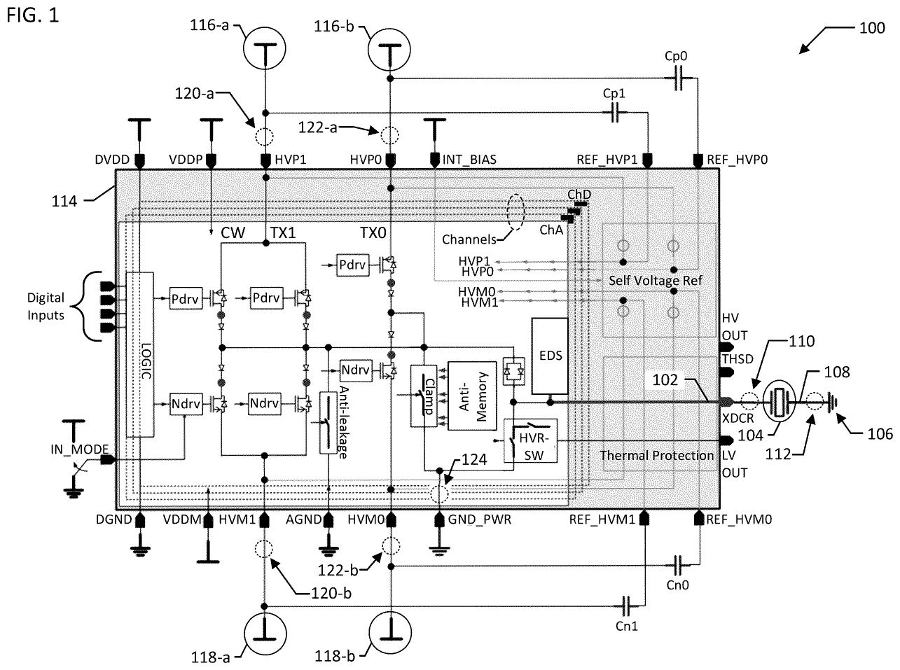

FIG. 1 an exemplary block diagram of a system 100 for detecting failed piezoelectric transducers in ultrasound applications in accordance with at least one embodiment of the present disclosure. The system 100 may include a piezoelectric driver circuit (e.g., piezoelectric driver circuitry 114) coupled to one or more piezoelectric elements (e.g., a piezoelectric element 104). The piezoelectric driver circuitry 114 may also be referred to as a piezoelectric driver, piezo driver circuitry, or a pulser. In some embodiments, the system 100 may be referred to as a pulser system (e.g., including pulser circuitry). In at least one embodiment, the system 100 may be embodied in an ultrasound pulser, such as an electrostatic discharge (ESD) enhanced high-speed ultrasound pulser. In some embodiments, the system 100 may be embodied in a monolithic, high-voltage, high-speed pulser generator that features multiple (independent) channels. As illustrated in the example of FIG. 1, the system 100 may include four channels (channel A (ChA) through channel D (ChD)). In some embodiments, the system 100 may be configured for ultrasound applications, such as medical ultrasound imaging applications. Additionally, or alternatively, the system 100 may be configured to drive piezoelectric, capacitive or micro-electromechanical systems (MEMS) based transducers.

The system 100 may support one or more features, including recirculation current protection, a relatively wide range of output voltages (e.g., 0 to ±90 V output voltage), an operating frequency up to about 20 megahertz (MHz) or some other suitable frequency, embedded low-power, floating high-voltage drivers and/or external voltage rails, one or more operation modes integrated claiming-to-ground function (e.g., with about an 8 Ohm (Ω) synchronous active clamp and anti-leakage circuitry), a dedicated half-bridge for continuous wave operations (denoted CW), an integrated transmission/reception switch (e.g., including about a 13Ω on-resistance, an high voltage (HV) metal oxide semiconductor (MOS) topology to reduce current consumption, up to about a 300 MHz bandwidth, and a receiver multiplexing function), and/or a complementary metal oxide semiconductor (CMOS) logic interface (e.g., about a 1.8 V to 3.6 V CMOS logic interface, and/or one or more auxiliary integrated circuits). The one or more operation modes may include a 3/5-level output waveform mode, a source and sink current mode, reduced jitter mode (e.g., with ≤20 picosecond (ps) jitter), an anti-cross conduction function mode, and/or a low 2^{nd} harmonic distortion mode. The one or more auxiliary integrated circuits may include noise blocking diodes, self-biasing architecture, anti-memory effect for internal HV nodes, thermal protections, standby function, and/or reinforced diodes on HV outputs for protection by recirculation current. The system 100 includes one or more HV power supplies for the piezoelectric driver circuitry 114, in which each HV power supply includes a positive terminal (e.g., a positive terminal 116-a or a positive terminal 116-b), and one or more corresponding negative terminals (e.g., a negative terminal 118-a and a negative terminal 118-b). The system 100 may also include one or more other components, such as one or more capacitors (denoted Cp1, Cp0, Cn1, and Cn0), among other components, to facilitate one or more functionalities of the system 100.

As illustrated in the example of FIG. 1, the system 100 (e.g., the piezoelectric driver circuitry 114 of the system 100) may include a controller logic interface circuit (denoted LOGIC), one or more level translators, one or more gate drivers (e.g., P-channel and N-channel metal-oxide-semiconductor field-effect transistor (MOSFET) gate drivers denoted Pdrv and Ndrv, respectively), one or more diodes (e.g., noise blocking diodes), and one or more high-power P-channel and N-channel MOSFETs (e.g., as the output stage for each channel). The system 100 may also include clamping-to-ground circuitry (denoted clamp), anti-leakage circuitry (denoted (anti-leakage), an anti-memory effect block (denoted anti-memory), one or more thermal sensors, and an HV receiver switch (denoted HVR-SW). The HV receiver switch may provide improved decoupling during the transmission phase. In some embodiments, the system 100 may include self-biasing circuitry (denoted self voltage ref) and/or one or more thermal shutdown blocks (denoted thermal protection).

Additionally, as illustrated in the example of FIG. 1, the system 100 may include one or more pins, such as one or more digital input pins (denoted digital inputs), a digital supply pin (denoted DVDD), a digital ground pin (denoted DGND), a positive supply voltage pin (denoted VDDP), a negative supply voltage pin (denoted VDDM), multiple HV positive supply pins (denoted HVP1 and HVP0), multiple HV negative supply pins (denoted HVM1 and HVM0), an analog ground pin (denoted AGND), multiple HV positive reference pins (denoted REF_HVP1 and REF_HVP0), multiple HV negative reference pins (denoted REF_HVM1 and REF_HVM0), a ground pin for the power supplies (denoted GND_PWR), one or more HV output pin (denoted HVOUT and XDCR), and/or a low voltage output pin (LVOUT). In some embodiments, the LVOUT pin is coupled to a low-noise amplifier (not shown). The system 100 may also include one or more dedicated pins, such as a thermal shutdown pin (denoted THSD) and a bias pin (denoted INT_BIAS). The bias pin may be used for determining whether a self-voltage reference is supplied to the piezoelectric driver circuitry 114 (e.g., via the REF_HVP1/REF_HVM1 and/or REF_HVP0/REF_HVM0 pins). In some embodiments, the system 100 includes a pin (denoted IN_MODE), which selects an interface control voltage level to the piezoelectric driver circuitry 114 (e.g., the pulser driver IC). In some embodiments, the interface control voltage level is selected between a nominal 1.8 V or 3.3 V interface.

In some embodiments, one or more channels (e.g., each channel) of the system 100 support up to five active output levels with two half bridges. For example, the system 100 may include a half-bridge (TX1) supplied by HVP1 and HVM1 and another half-bridge (TX0) supplied by HVP0 and HVM0. In some embodiments, the DVDD and VDDP pins may operate at about 3.3V, the VDDM pin may operate at about −3.3V (or another suitable voltage), HVP1 and HVP0 pins may operate at 0 to about 90V (or another suitable voltage), HVM1 and HVM0 may operate at 0 to about −90V (or another suitable voltage), INT_BIAS pin may operate at about 3V (or another suitable voltage), IN_MODE pin may operate at about 3.3V (or another suitable voltage), and the digital input pins may operate at about 1.8V to about 3.3V (or another suitable voltage). In some embodiments, the output stage of a channel (e.g., each channel) may provide a peak output current of about ±2 A (or another suitable amperage). To reduce power dissipation, such as during continuous wave mode, the system 100 may include a dedicated half-bridge (denoted CW) in which the peak current may be constrained to about 0.6 A (or another suitable amperage).

In some embodiments, the system 100 includes an ESD reinforced structure to prevent high voltage overshoots and inrush current due to cabled transducer reflections. The system 100 includes multiple HV pins (e.g., HVP1, HVM1, HPV0, and HVM0). The HV pins (e.g., each HV output) are supported by a dedicate HV diode network to withstand possible reflections, mitigating excessive voltage overshoot that could lead to device damage.

In some embodiments, the system 100 (e.g., a piezoelectric driver integrated circuit) is configured to convert relatively low voltage to relatively high voltage and then sequentially apply the relatively high voltage to one or more piezoelectric elements (e.g., each of the piezoelectric elements). In some such embodiments, the system 100 may be configured to apply the high voltage to an array of multiple piezoelectric elements (e.g., 56 piezoelectric elements). For example, t he system 100 may be configured to output a signal (e.g., current) via the XDCR pin to the piezoelectric element 104 (e.g., a piezoelectric transducer) via a forward path 102. The piezoelectric element 104 is coupled, via a return path 108, to a ground pin 106. Accordingly, in the system 100, current may flow in the forward path 102 to the piezoelectric element 104 and in the return path 108 from the piezoelectric element 104.

In some embodiments, if a piezoelectric element is damaged the current consumption of the damaged piezoelectric elements may be different (e.g., will be substantially different) from one or more other undamaged piezoelectric element. Accordingly, the system 100 may be configured to measure the current consumed by the piezoelectric elements (e.g., each of the 56 piezoelectric elements) to identify failed piezoelectric elements. That is, as illustrated in the example of FIG. 1, the system 100 may be configured to support one or more mechanisms for detecting failed piezoelectric transducers. Because the impedance of an intact piezoelectric element (e.g., a piezoelectric crystal transducer) is constant, the current at a fixed voltage, frequency and temperature will also be constant. This current may be measured (e.g., repeatedly) and, for an undamaged piezoelectric element, will be the same within the tolerances of the system, in which the tolerances of the system may be based on the driver circuit, the measurement circuit, the interface PCB and/or the flex circuit (or other transmission medium) to the piezoelectric element.

In some embodiments, the system 100 may be modified to include current sense circuitry (e.g., a sensor and associated circuitry) at one or more locations within the system 100. That is, the system 100 may be modified to add one or more current sensing elements to the system 100, to determine the current consumed by one or more piezoelectric elements. In other words, the system 100 may include current sense circuitry positioned to sense current at one or more locations within the system 100.

In some embodiments, the system 100 may be modified at one or more locations to derive the current consumption by the piezoelectric element 104. In some such embodiments, the current may be measured directly at the piezoelectric transducer output stage for the piezoelectric element 104. For example, the system 100 may include current sense circuitry in the forward path 102, such as at a first location 110 (or another suitable location) between the XDCP pin and the piezoelectric element 104. Additionally, or alternatively, the current may be measured at the return path to ground from the piezoelectric element 104. For example, the system 100 may include current sense circuitry at a second location 112 (or another suitable location) in the return path 108. Additionally, or alternatively, the current may be measured at one or more HV power supplies (e.g., HVP1 and HVM1 and/or HVP0 and HVP1) and/or at the respective return path to ground from the HV power supplies. For example, the system 100 may include current sense circuitry at third locations 120 (e.g., a third location 120-a for HVP1 and a third location 120-b for HVM1) and/or at fourth locations 122 (e.g., a fourth location 122-a for HVP0 and a fourth location 122-b for HVM0). While the third locations 120 and the fourth locations 122 are positioned outside of the piezoelectric driver circuitry 114 in the example of FIG. 1, it should be appreciated that the current circuitry may be positioned inside of the piezoelectric driver circuitry 114. Additionally, or alternatively, the system 100 may include current sense circuitry at the GND_PWR pin to measure return currents. For example, the system 100 may also include current sense circuitry at a fifth location 124 (or another suitable location) in the return path to the GND_PWR pin from the HV power supplies. While the fifth location 124 is positioned inside of the piezoelectric driver circuitry 114 in the example of FIG. 1, it should be appreciated that the current circuitry may be positioned outside of the piezoelectric driver circuitry 114. In some embodiments, the system 100 may determine to measure return currents (e.g., via the current sense circuitry at the GND_PWR pin) in the event of a failed piezoelectric element, which may have become inductive in nature.

Although the example of FIG. 1 illustrates particular locations for current measurements (e.g., the first location 110, the second location 112, the third locations 120, the fourth locations 122, and the fifth location 124), it should be understood that these locations are merely examples and should not be construed as limitations on the scope of the disclosure or of what may be claimed.

In other words, the current consumed by the piezoelectric element 104 may be measured at one or more of several different locations to provide an indication of piezoelectric element integrity, in which the different locations include the piezoelectric driver power supply source and ground paths, as well as the piezoelectric element power source and ground paths. Current measurements on the ground path of the piezoelectric element (e.g., at the second location 112) may provide several benefits. For example, current measurements at the ground path reflect (relatively precisely) the current consumed by the piezoelectric element 104 and exclude power losses associated with high voltage power supply management and control circuitry driving the piezoelectric element. Additionally, current measurements in the ground path of the piezoelectric element 104, provide for a low common mode voltage due to the current measurements being taken relatively close to circuit ground. Current measurements in the ground path may therefore allow for improved interfacing to an on-board low voltage interface/amplifying circuit, which provides the current measurement information to other circuity. Such other circuitry may include circuitry associated with determining whether the current measurement information indicates the presence of a failed piezoelectric element. Measuring current at the piezoelectric element source (e.g., at the first location 110), may place the common mode voltage near the high voltage power supply rail and, as such, a voltage interface/amplifying circuitry may be constrained to operating at voltages that are not suitable for the voltage interface/amplifying circuitry (e.g., are unusual for that type of circuitry). Current measurements at the piezoelectric element ground path (e.g., at the second location 112) result in a lower common mode voltage, which is suitable for the voltage interface/amplifying circuitry. In other words, by measuring the current at the second location 112, the system 100 may enable a common mode volage that is usual and customary in one or more types of voltage interface/amplifying circuitry.

In some embodiments, the method to derive current may be based on a shunt resistor, a Hall Effect technique, a GMR technique and/or a TMR technique. In some instances, the method to derive the current may depend on the location at which the current is measured. For example, because of the high voltage present in the power supply to the piezoelectric driver circuitry 114 (e.g., the high voltage present at HVP1/HVM1 and/or HVP0/HVM0), implementation of a shunt resistor may present common mode voltages that may exceed the process technology utilized to measure the current and/or the piezoelectric element itself. In some instances, isolation may be achieved by using hall effect current sensors and/or by isolating the high voltage present to the measurement circuit. Additionally, or alternatively, the current may be measured at the piezoelectric element ground path, which results in a lower common mode voltage and improves the resolution and accuracy of the current measurements.

In some embodiments, the system 100 may be configured to perform current consumption validation. For example, in some such embodiments, the system 100 may be configured to obtain (and store) baseline current consumption measurements for current consumption validation of the piezoelectric element 104. Subsequent to obtaining the baseline current measurements, the system 100 may be configured to perform current consumption validation by measuring the current consumed by the piezoelectric element 104, comparing the subsequent measurements against the baseline measurements, and determining whether the subsequent measurements are within the system tolerance (e.g., are consistent with the baseline performance of the piezoelectric element 104).

In some embodiments, the system 100 may be configured to perform one or more current consumption measurements as part of a self-test, a power-on routine, and/or in response to a command. Additionally, or alternatively, in some embodiments, the system 100 may be configured to obtain one or more baseline current measurements periodically (e.g., according to a preconfigured periodicity) and/or in response to a command. In some embodiments, if a current measurement of a piezoelectric element deviates from the baseline measurement (e.g., by an amount that is outside of the system tolerance), the system 100 may determine the piezoelectric element 104 (or a transmission medium associated with the piezoelectric element 104, such as a cable connected to the piezoelectric element 104) is damaged or otherwise failed. In some such embodiments, the system 100 may be configured to output a message (e.g., an alert) indicating that the piezoelectric element is damaged or otherwise failed. For example, the system 100 (or other associated circuitry) may be configured to output a message indicating that the piezoelectric element is operating in a sub-performance state.

In some embodiments, the system 100 may be configured to obtain (and record) the baseline (normal) current at a fixed high voltage, fixed frequency, with known transducer characteristics, and with a known transmission medium to the transducer, such as a flex circuit or cable. Additionally, in some embodiments, the system 100 may be configured to obtain the baseline current at a range of ambient temperatures. In some instances, subtle differences associated with piezoelectric element placement, such as within an array, may impact a baseline current measurement. Accordingly, the system 100 may be configured to measure the baseline current for one or more piezoelectric elements. In some embodiments, the system 100 may be configured to record a respective baseline measurement for each piezoelectric elements in the array.

In some embodiments, after the baseline current is recorded for one or more piezoelectric elements, a suitable system tolerance may be determined. The system tolerance may depend on the piezoelectric element itself, a lifetime drift of the piezoelectric element, a variation expected for ambient temperatures, and one or more other system drift components. The system tolerance (e.g., a respective system tolerance for each piezoelectric element) may be stored within the system 100 (or another associated system) for later use. For example, the system 100 may store the system tolerance (e.g., information associated with the system tolerance) for one or more current consumption validation events. In some embodiments, the system 100 may use the system tolerance to determine a threshold amperage associated with one or more baseline current consumption measurements for the piezoelectric element 104. In some such examples, the system 100 may determine that a current consumption measurement that deviates from the one or more baseline current consumption measurements by an amount that is less than the threshold is within the system tolerance. In some other examples, the system 100 may determine that a current consumption measurement that deviates from the one or more baseline current consumption measurements by an amount that is greater than the threshold is outside of the system tolerance.

In some embodiments, the system 100 may utilize one or more interface ports to communicate interrupt/interrogate events, such as current consumption validation. In some embodiments, the system 100 may be configured to perform a comparison of the baseline current measurement per piezoelectric element, to a subsequent (e.g., newly acquired) current measurement in response to a command, which may be initiated based on one or more conditions, such as power up, drop detection, or time interval (e.g., to assure correct operation), among other examples of events that may trigger a measurement command. After a comparison of the baseline current to the newly measured current, the system 100 may determine whether a difference between the baseline current and the newly measurement current satisfies the system tolerance (e.g., predefined pass/fail criteria).

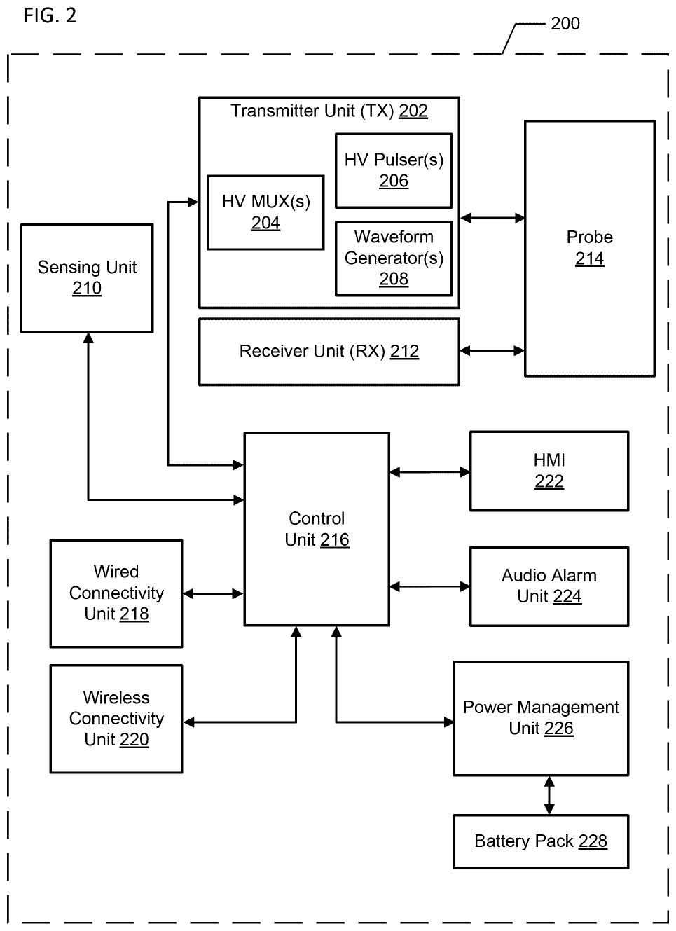

FIG. 2 illustrates an exemplary block diagram of an ultrasound system 200 configured for detecting failed piezoelectric transducers in ultrasound applications in accordance with at least one embodiment of the present disclosure. Ultrasound imaging works by using a probe to directly apply sound waves (e.g., in the non-audible spectrum) to a surface of an object and analyzing the different travel times and amplitudes generated by echoes of the sound waves, in response to the sound waves interacting with different parts of the object. For example, an ultrasound tool may directly apply sound waves to a patient's skin and analyze the different travel times and amplitudes generated by echoes of the sound waves, in response to the sound waves interacting with different tissues and organs in the patient. Ultrasound tools are non-destructive and provide real-time images, which can be used for various applications, such as for echocardiograms and fetal diagnostics. The quality of the reconstructed image is highly dependent upon the probe, which typically uses piezoelectric transducers.

In some instances, it may be relatively difficult to determine whether a piezoelectric transducer in an ultrasound tool is damaged. For example, some methods for detecting damaged piezoelectric transducers include a user identifying a failure of the ultrasound tool through use of the ultrasound tool, such as during a patient procedure. Consequently, the user may not know that a piezoelectric transducer in an ultrasound tool has been damaged (e.g., from a dropping incident) prior to using the ultrasound tool and/or until an image is displayed and an anomaly is observed within the image, which may lead to delays for the patient and/or a misdiagnosis.

The ultrasound system 200 may provide for improved detection of failed piezoelectric transducers in accordance with one or more systems and methods described herein. As illustrated in the example of FIG. 2, the ultrasound system 200 includes a control unit 216, as well as a transmitter unit 202, a sensing unit 210, a wired connectivity unit 218, a wireless connectivity unit 220, a human sensing interface (HMI) 222, a power management unit 226, and an audio alarm unit 224, all of which are coupled to the control unit 216. In some embodiments, the transmitter unit 202 may include one or more HV multiplexers 204 (denoted HV MUX(s)), one or more HV pulser(s), and one or more waveform generators 208. The transmitter unit 202 may be coupled to a probe 214, which may be coupled to a receiver unit 212. The probe 214 may include one or more piezoelectric transducer elements. For example, the probe 214 may include a piezoelectric bulk array (e.g., a lead zirconate titanate (PZT) bulk array) and/or a piezoelectric micromachined ultrasonic transducer (pMUT) array. In some embodiments, the probe may include one or more components configured for probe authentication. The one or more piezoelectric transducer elements may be examples of a piezoelectric element 104 illustrated by and described with reference to FIG. 1. Additionally, the one or more HV pulsers 206 included in the transmitter unit 202 may be examples of a piezoelectric driver circuitry 114 illustrated by and described with reference to FIG. 1. In some embodiments, the one or more HV pulsers 206 (e.g., at one or more XDCR pins) within the transmitter unit 202 may be coupled to the one or more piezoelectric transducer elements within the probe 214.

The sensing unit 210 may include one or more temperature sensors, one or more accelerometers, one or more inertial modules, and/or a MEMS audio sensor (e.g., microphone). The control unit may include one or more microcontroller units (MCUs), a delay generator (e.g., a field programmable gate array (FPGA)), and one or more components configured to support embedded security and EDS protection. The wired connectivity unit 218 may include one or more types of ports (e.g., a USB type C port, an ethernet port) and one or more components to support protections for the one or more ports (e.g., ethernet protection, USB protection). In some embodiments, the wired connectivity unit 218 may be configured to support physical (PHY) layer protocols, such as ethernet PHY protocols. The wireless connectivity unit 220 may include one or more components to support WiFi communication protocols, Bluetooth communication protocols (e.g., Bluetooth low-energy), one or more baluns, one or more filters, and one or more components configured to support antenna protection.

The HMI 222 may include a display backlight (e.g., a liquid-crystal display backlight), one or more light emitting diode (LED) drivers, one or more audio amplifiers, and one or more components configured to support backlight protection and audio protection. The audio alarm unit 224 may include one or more audio amplifiers and/or one or more audio protections. The power management unit 226 may be coupled to a battery pack 228. The power management unit 226 may include one or more buck regulators, one or more voltage regulators, one or more components configured to support protection for the one or more buck regulators and/or the one or more voltage regulators, and/or a power Schottky diode. The battery pack 228 may include one or more battery management ICs, one or more components to support a battery management system, and one or more batteries. In some embodiments, one or more components of the ultrasound system 200 may be included in a mobile device (e.g., a handheld device that is portable).

In some embodiments, the ultrasound system 200 may be configured to determine the current consumed by one or more piezoelectric elements within the probe 214. For example, a piezoelectric element within the probe 214 may be coupled to the HV pulser 206 (e.g., a piezoelectric driver circuit), such that the HV pulser 206 may serve as a current source for the piezoelectric element. For example, the piezoelectric element may draw current from the HV pulser 206 to generate one or more ultrasonic signals. In accordance with one or more embodiments, the current consumed by the piezoelectric element may be measured in a return path from the one or more piezoelectric elements. For example, the ultrasound system 200 may be configured to determine a current consumed by the piezoelectric element via a sensor positioned to sense current in a return path from the piezoelectric element. Additionally, the ultrasound system 200 may be configured to determine an operational state of the piezoelectric element based on the current consumed by the piezoelectric element. In some embodiments, the ultrasound system 200 may determine that the operational state of the piezoelectric element is a sub-performance state based on the current failing to satisfy one or more criteria associated with a baseline performance of the piezoelectric element. In some examples, the ultrasound system 200 may be configured to output an alert (e.g., via the audio alarm unit 224) in response to determining that the operational state of the piezoelectric element is a sub-performance state. In some embodiments, the ultrasound system 200 (e.g., via the control unit 216) may trigger the ultrasound system 200 to determine a current consumed by the piezoelectric element (e.g., may trigger current consumption validation) as part of a self-test or a power-on routine, among other examples. In some examples, by sensing the current in the return path of the piezoelectric element, the ultrasound system 200 may provide for improved resolution and accuracy of current measurements, among other benefits.

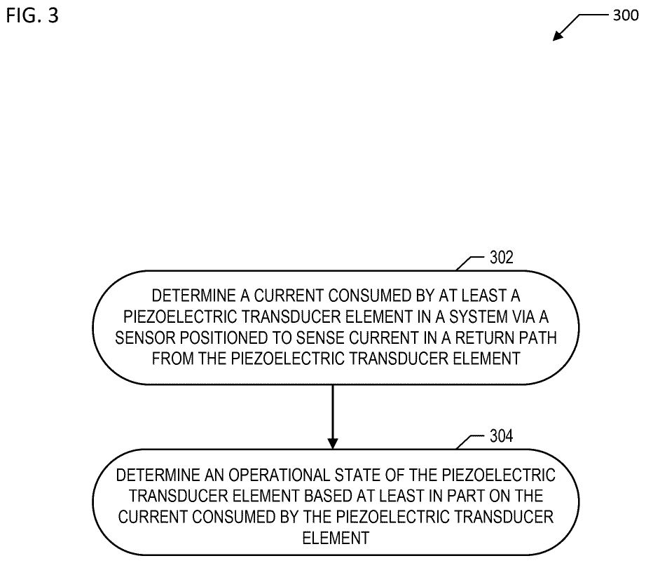

FIG. 3 illustrates an exemplary flowchart 300 of operations that support systems and methods for detecting failed piezoelectric transducers in accordance with at least one embodiment of the present disclosure. FIG. 3 may be implemented by one or more aspects illustrated by and described with reference to FIGS. 1 and 2. For example, the operations illustrated in FIG. 3 may be implemented by a system, which may be an example of the system 100 or the ultrasound system 200 illustrated by and described with reference to FIG. 1 and FIG. 2, respectively.

At operation 302, the system may determine a current consumed by at least a piezoelectric transducer element in a system via a sensor positioned to sense current in a return path from the piezoelectric transducer element. In some embodiments, the return path includes an electrical path between the piezoelectric transducer element and a ground terminal. In some embodiments, the sensor is a shunt resistor (e.g., a sense resistor), a Hall effect sensor, a GMR sensor, or a TMR sensor.

At operation 304, the system may determine an operational state of the piezoelectric transducer element based on the current consumed by the piezoelectric transducer element. In some embodiments, the system may determine that the operational state of the piezoelectric transducer element is a sub-performance state based on the current failing to satisfy one or more criteria associated with a baseline performance of the piezoelectric transducer element. For example, the system may be configured to compare the current consumed by the piezoelectric transducer element to a predicted current consumption for the piezoelectric transducer element and may determine that a difference between the value of the current and the value of a predicted current consumption is greater than a threshold.

In some embodiments, the predicted current consumption is based on one or more baseline current measurements and/or the system tolerance. For example, in some embodiments, the system may obtain one or more baseline current measurements associated with the piezoelectric transducer element. In some such embodiments, the one or more criteria (e.g., the predicted current and/or the threshold) are based on the one or more baseline current measurements.

In some embodiments, the system is configured to determine the system tolerance based on the one or more baseline current measurements. For example, in some embodiments, the system may be configured to use the one or more baseline current measurements to determine a lifetime drift and an expected temperature variation associated with current consumption by the piezoelectric transducer element. In some such embodiments, the one or more criteria (e.g., the predicted current and/or the threshold) are based on the lifetime drift and the expected temperature variation. In some embodiments, the one or more criteria (e.g., the system tolerance) are further based on an interface associated with the piezoelectric transducer element (e.g., an interface PCB and/or flex circuit or cable) and/or a position of the piezoelectric transducer element within an array (e.g., a PZT bulk array).

In some embodiments, the piezoelectric transducer element is one of a multiple piezoelectric transducer elements within the system. In some such embodiments, the system may determine a respective current consumed by each piezoelectric transducer element of the multiple piezoelectric transducer elements via one or more sensors positioned to sense current in one or more return paths from the multiple piezoelectric transducer elements, in which the one or more sensors include at least the sensor and the one or more return paths include at least the return path.

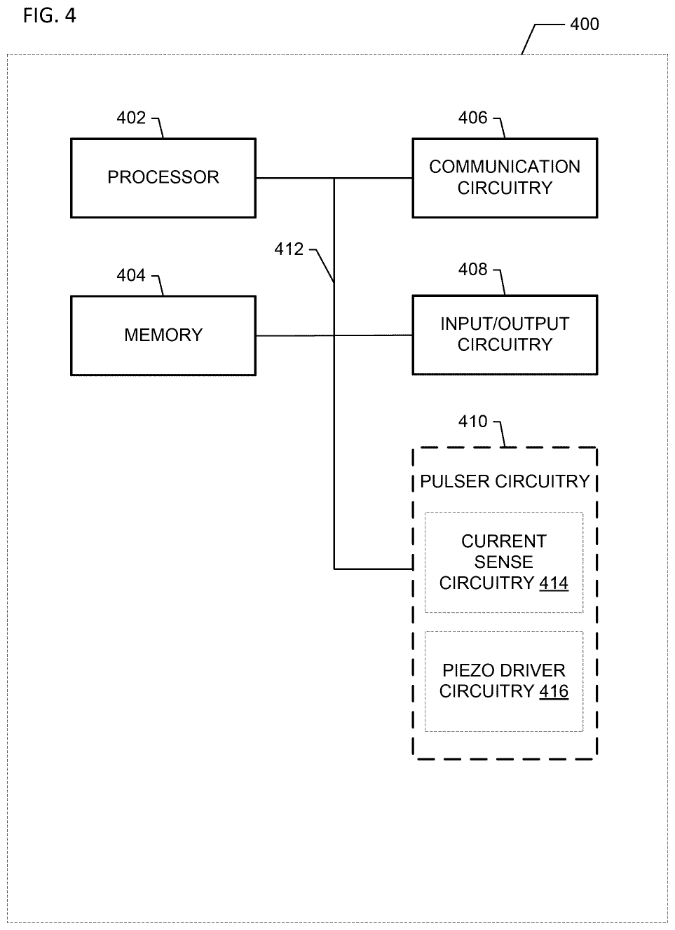

FIG. 4 illustrates an exemplary device 400 that support systems and methods for detecting failed piezoelectric transducers in accordance with at least one embodiment of the present disclosure. FIG. 4 may implement one or more aspects illustrated by and described with reference to FIGS. 1, 2, and 3. The device 400 may be a device for an application, apparatus, and/or a system described herein. For example, the device 400 may be, or be implemented in an ultrasound device or a device for another application, such those described herein.

The device 400 may be a system and/or apparatus that includes a processor 402, memory 404, communication circuitry 406, input/output circuitry 408, pulser circuitry 410, and all of which may be connected by a bus or buses 412. It should be appreciated that, in some embodiments, the device 400 may include or be otherwise coupled to one or more other components, such as a power source, a load(s), and/or a controller for one or more switches. The power source, controller, and/or load(s) may be internal or external to the device 400. For example, the power source, load, and/or controller may be coupled to at least the pulser circuitry 410 via a bus or one or more connectors.

The processor 402, although illustrated as a single block, may be comprised of a plurality of components and/or processor circuitry. The processor 402 may be implemented as, for example, various components comprising one or a plurality of microprocessors with accompanying digital signal processors; one or a plurality of processors without accompanying digital signal processors; one or a plurality of coprocessors; one or a plurality of multi-core processors; processing circuits; and various other processing elements. The processor may include integrated circuits. In some embodiments, the processor 402 may be configured to execute applications, instructions, and/or programs stored in the processor 402, or otherwise accessible to the processor 402. When executed by the processor 402, these applications, instructions, and/or programs may enable the execution of one or a plurality of the operations and/or functions described herein. Regardless of whether it is configured by hardware, firmware/software methods, or a combination thereof, the processor 402 may comprise entities capable of executing operations and/or functions according to the embodiments of the present disclosure when correspondingly configured. In some embodiments, the processor 402 may be configured to determine (e.g., in combination with the pulser circuitry 410 and/or the memory 404) an operational state of the piezoelectric transducer element.

The memory 404 may comprise, for example, a volatile memory, a non-volatile memory, or a certain combination thereof. Although illustrated as a single block, the memory 404 may comprise a plurality of memory components. In some embodiments, the memory 404 may comprise, for example, a random access memory, a cache memory, a flash memory, a hard disk, a circuit configured to store information, or a combination thereof. The memory 404 may be configured to write or store data, information, application programs, instructions, etc. so that the processor 402 may execute various operations and/or functions according to the embodiments of the present disclosure. For example, in at least some embodiments, a memory 404 may be configured to buffer or cache data for processing by the processor 402. Additionally, or alternatively, in at least some embodiments, the memory 404 may be configured to store program instructions for execution by the processor 402. The memory 404 may store information in the form of static and/or dynamic information. When the operations and/or functions are executed, the stored information may be stored and/or used by the processor 402. In some embodiments, the memory 404 may be configured to store information pertaining to a respective baseline performance of one or more piezoelectric elements. For example, the memory 404 may be configured to store information pertaining to a predicted current for the one or more piezoelectric elements and/or a threshold associated with the system tolerance. Additionally, or alternatively, the memory 404 may be configured to store information pertaining to one or more baseline current measurements associated with the one or more piezoelectric elements, a lifetime drift and an expected temperature variation associated with current consumption by the one or more piezoelectric elements, and/or one or more characteristics associated with the pulser circuitry 410 that may impact a baseline current performance associated with the one or more piezoelectric elements.

The communication circuitry 406 may be implemented as a circuit, hardware, computer program product, or a combination thereof, which is configured to receive and/or transmit data from/to another component or apparatus. The computer program product may use computer-readable program instructions stored on a computer-readable medium (e.g., memory) and executed by a processor 402. In some embodiments, the communication circuitry 406 (as with other components discussed herein) may be at least partially implemented as part of the processor 402 or otherwise controlled by the processor 402. The communication circuitry 406 may communicate with the processor 402, for example, through a bus 412. Such a bus 412 may connect to the processor 402, and it may also connect to one or more other components of the processor 402. The communication circuitry 406 may be comprised of, for example, transmitters, receivers, transceivers, network interface cards and/or supporting hardware and/or firmware/software and may be used for establishing communication with another component(s), apparatus(es), and/or system(s). The communication circuitry 406 may be configured to receive and/or transmit data that may be stored by memory by using one or more protocols that can be used for communication between components, apparatuses, and/or systems. In some embodiments, the communication circuitry 406 may be configured to output a message (e.g., an alert) to a user of the device 400 in response to detecting a failed piezoelectric transducer element. For example, the communication circuitry 406 may be configured to output a message (e.g., an alert) to a user of the device 400 in response to determining that the operational state of a piezoelectric element is a sub-performance state based on the current failing to satisfy one or more criteria associated with a baseline performance of the piezoelectric element.

The input/output circuitry 408 may communicate with the processor 402 to receive instructions input by an operator and/or to provide audible, visual, mechanical, or other outputs to an operator. The input/output circuitry 408 may comprise supporting devices, such as a keyboard, a mouse, a user interface, a display, a touch screen display, lights (e.g., warning lights), indicators, speakers, and/or other input/output mechanisms. The input/output circuitry 408 may comprise one or more interfaces to which supporting devices may be connected. In some embodiments, aspects of the input/output circuitry 408 may be implemented on a device used by the operator to communicate with the processor 402. The input/output circuitry 408 may communicate with memory, the communication circuitry 406, and/or any other component, for example, through a bus 412.

The pulser circuitry 410 may be an example of a system 100 illustrated by and described with reference to FIG. 1. For example, the pulser circuitry 410 may include current sense circuitry 414 (e.g., a sensor and associated circuitry), which may be an example of current sense circuitry described with reference to FIGS. 1 through 3. The pulser circuitry 410 may also include at least a piezoelectric element configured to receive a current signal from (i.e., draw current from) a piezo driver circuitry 416, which may be an example of a piezoelectric driver circuit illustrated by and described with reference to FIG. 1. The piezoelectric element may be configured to convert the current drawn by the piezoelectric transducer element (or a portion thereof) into one or more signals. In other words, current from the piezoelectric driver circuit may be consumed by the piezoelectric element for generation of one or more signals. The pulser circuitry 410 may be configured to determine (i.e., sense) a current consumed the piezoelectric transducer element via the current sense circuitry 414. In some embodiments, the current sense circuitry 414 is positioned to sense the current in a return path from the piezoelectric transducer element. The current sense circuitry 414 may be coupled to the piezo driver circuitry 416 and/or the piezoelectric transducer element. The pulser circuitry 410 may be configured to determine (e.g., in combination with the processor 402 and/or the memory 404) an operational state of the piezoelectric transducer element based on the current consumed by the piezoelectric transducer element.

The device 400 may be implement in hardware, software, or a combination of hardware and software. In some embodiments, the device 400 may be embodied in an integrated circuit, a microcontroller unit (MCU) (e.g., virtual machine running in an MCU), and/or the like. It should be readily appreciated that the embodiments of the systems, apparatuses, and methods described herein may be configured in various additional and alternative manners in addition to those expressly described herein.

### Conclusion

Operations and/or functions of the present disclosure have been described herein, such as in flowcharts. As will be appreciated, computer program instructions may be loaded onto a computer or other programmable apparatus (e.g., hardware) to produce a machine, such that the resulting computer or other programmable apparatus implements the operations and/or functions described in the flowchart blocks herein. These computer program instructions may also be stored in a computer-readable memory that may direct a computer, processor, or other programmable apparatus to operate and/or function in a particular manner, such that the instructions stored in the computer-readable memory produce an article of manufacture, the execution of which implements the operations and/or functions described in the flowchart blocks. The computer program instructions may also be loaded onto a computer, processor, or other programmable apparatus to cause a series of operations to be performed on the computer, processor, or other programmable apparatus to produce a computer-implemented process such that the instructions executed on the computer, processor, or other programmable apparatus provide operations for implementing the functions and/or operations specified in the flowchart blocks. The flowchart blocks support combinations of means for performing the specified operations and/or functions and combinations of operations and/or functions for performing the specified operations and/or functions. It will be understood that one or more blocks of the flowcharts, and combinations of blocks in the flowcharts, can be implemented by special purpose hardware-based computer systems which perform the specified operations and/or functions, or combinations of special purpose hardware with computer instructions.

While this specification contains many specific embodiments and implementation details, these should not be construed as limitations on the scope of any disclosures or of what may be claimed, but rather as descriptions of features specific to particular embodiments of particular disclosures. Certain features that are described herein in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

While operations and/or functions are illustrated in the drawings in a particular order, this should not be understood as requiring that such operations and/or functions be performed in the particular order shown or in sequential order, or that all illustrated operations be performed, to achieve desirable results. In certain circumstances, operations and/or functions in alternative ordering may be advantageous. In some cases, the actions recited in the claims may be performed in a different order and still achieve desirable results. Thus, while particular embodiments of the subject matter have been described, other embodiments are within the scope of the following claims.

While this detailed description has set forth some embodiments of the present invention, the appended claims cover other embodiments of the present invention which differ from the described embodiments according to various modifications and improvements.

Within the appended claims, unless the specific term “means for” or “step for” is used within a given claim, it is not intended that the claim be interpreted under 35 U.S. C. § 112, paragraph 6.

1. A method comprising:
determining a current consumed by at least a piezoelectric transducer element in a system via a sensor positioned to sense current in a return path from the piezoelectric transducer element; and
determining an operational state of the piezoelectric transducer element based at least in part on the current consumed by the piezoelectric transducer element.

2. The method of claim 1, wherein determining the operational state of the piezoelectric transducer element comprises:
determining that the operational state of the piezoelectric transducer element is a sub-performance state based at least in part on the current failing to satisfy one or more criteria associated with a baseline performance of the piezoelectric transducer element.

3. The method of claim 2, further comprising:
obtaining one or more baseline current measurements associated with the piezoelectric transducer element, wherein the one or more criteria are based at least in part on the one or more baseline current measurements.

4. The method of claim 3, wherein a baseline current measurement of the one or more baseline current measurements comprise a current measurement obtained at a fixed voltage, a fixed operating frequency, and a fixed temperature.

5. The method of claim 4, further comprising:
determining, based at least in part on the one or more baseline current measurements, a lifetime drift and an expected temperature variation associated with current consumption by the piezoelectric transducer element, wherein the one or more criteria are based at least in part on the lifetime drift and the expected temperature variation.

6. The method of claim 2, wherein the one or more criteria are further based at least in part on at least one of: an interface associated with the piezoelectric transducer element or a position of the piezoelectric transducer element within an array.

7. The method of claim 1, wherein the return path comprises an electrical path between the piezoelectric transducer element and a ground terminal.

8. The method of claim 1, wherein the sensor comprises a shunt resistor, a Hall effect sensing device, a giant magneto resistance (GMR) sensing device, or a tunnel magneto resistance (TMR) sensing device.

9. The method of claim 8, wherein the sensor comprises the Hall effect sensing device.

10. The method of claim 1, wherein the piezoelectric transducer element is one of a plurality of piezoelectric transducer elements within the system, and wherein the method further comprises:
determining a respective current consumed by each piezoelectric transducer element of the plurality of piezoelectric transducer elements via one or more sensors positioned to sense current in one or more return paths from the plurality of piezoelectric transducer elements, the one or more sensors comprising at least the sensor and the one or more return paths comprising at least the return path.

11. The method of claim 1, wherein the system comprises an ultrasound system.

12. A system comprising:
a piezoelectric transducer element;
a sensor positioned to sense current in a return path from the piezoelectric transducer element; and
a processor configured to determine an operational state of the piezoelectric transducer element based at least in part on the current sensed in the return path.

13. The system of claim 12, wherein the processor is further configured to determining that the operational state of the piezoelectric transducer element is a sub-performance state based at least in part on the current failing to satisfy one or more criteria associated with a baseline performance of the piezoelectric transducer element.

14. The system of claim 13, wherein the processor is further configured to obtain one or more baseline current measurements associated with the piezoelectric transducer element, wherein the one or more criteria are based at least in part on the one or more baseline current measurements.

15. The system of claim 12, wherein the return path comprises an electrical path between the piezoelectric transducer element and a ground terminal.

16. The system of claim 12, wherein the sensor comprises a shunt resistor, a Hall effect sensing device, a giant magneto resistance (GMR) sensing device, or a tunnel magneto resistance (TMR) sensing device.

17. An apparatus comprising:
at least one processor; and
at least one memory having computer program code stored thereon that, in execution with the at least one processor, causes the apparatus at least to:
determine a current consumed by at least a piezoelectric transducer element in a system via a sensor positioned to sense current in a return path from the piezoelectric transducer element; and
determine an operational state of the piezoelectric transducer element based at least in part on the current consumed by the piezoelectric transducer element.

18. The apparatus of claim 17, wherein the computer program code, in execution with the at least one processor, causes the apparatus to:
determine that the operational state of the piezoelectric transducer element is a sub-performance state based at least in part on the current failing to satisfy one or more criteria associated with a baseline performance of the piezoelectric transducer element.

19. The apparatus of claim 18, wherein the computer program code, in execution with the at least one processor, causes the apparatus to:
obtain one or more baseline current measurements associated with the piezoelectric transducer element, wherein the one or more criteria are based at least in part on the one or more baseline current measurements.

20. The apparatus of claim 17, wherein the return path comprises an electrical path between the piezoelectric transducer element and a ground terminal.
